(19) United States
(12) Patent Application Publication
(10) Pub. No.: US 2008/0308548 A1
(43) Pub. Date: Dec. 18, 2008

(54) Active Matrix Temperature Controller Array

(75) Inventors: David Andrew Fish, Haywards Heath (GB); Marc Wilhemus Gijsbert Ponjee, Eindhoven (NL); Mark Thomas Johnson, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 12/097,793

(22) PCT Filed: Dec. 15, 2006
(86) PCT No.: PCT/IB06/54893

§ 371 Date: Jun. 17, 2008

(30) Foreign Application Priority Data
Dec. 19, 2005 (EP) 05112373.5

(51) Int. Cl. G05D 23/20; C12M 1/34

(52) U.S. Cl. 219/494; 435/288.4

(57) ABSTRACT

In an array for temperature controlled cells the cells are driven in an active matrix array. A temperature processing array may be employed in biochip, such as underneath a biosensor or underneath reaction chambers. Due to the active matrix complex driver circuitry may be positioned outside the actual array of cells. Each cell is provided with a switch for coupling the cell circuitry to the driver circuitry. When coupled to the driver circuitry, a memory element in the cell circuitry may be provided with a heating setting. Then, the cell circuitry is uncoupled from the driver circuitry and a heating element is controlled to heat the cell in accordance with the setting stored in the memory element.

### FIELD OF THE INVENTION

The present invention relates to a method for driving an array of temperature controlled cells and to an array of temperature controlled cells.

### BACKGROUND OF THE INVENTION

An array of temperature controlled cells, also referred to as a temperature-processing array, is in general applied in devices in which the temperature of multiple cells is to be controlled independently from the temperature of other cells. An exemplary application is application in a bio-chip. Such a bio-chip may, for example, be suited to perform a chemical reaction. The cells may then represent a reaction chamber, whereby the cells may comprise a sealed or seal-able compartment, and the array may be employed for heating, and controlling the temperature, of each reaction chamber on the bio-chip. Alternatively, the cells may represent different regions in a larger compartment which need to be individually temperature controlled. Fluid may flow across or between the compartments.

An exemplary application is the use of a temperature-processing array for the thermal cycling during DNA amplification, such as Polymerase Chain Reaction (PCR). This is a temperature controlled and enzyme-mediated amplification technique for nucleic acid molecules, usually comprising a periodical repetition of three reaction steps: a denaturing step at about 92-96° C., an annealing step at about 37-65° C. and an extending step at about 72° C. The basic requirement for an efficient amplification is rapid heat transfer, which makes temperature control an important feature of a micro-PCR system.

US 2002/0048765 discloses an integrated micro-array device having a number of temperature controlled reaction wells. Each well may be provided with a temperature control chip. Each chip is individually driven through two addressing wires, resulting in a complex and thereby expensive design.

### OBJECT OF THE INVENTION

It is desirable to provide a simple and low-cost array of temperature controlled cells.

### SUMMARY OF THE INVENTION

In a first aspect the present invention provides a method of driving an array of temperature controlled cells, each cell comprising a heat control means comprising a heating element, a switch element, and a temperature sensor, and the array further comprising a driver circuit, the method comprising:

supplying an address signal for controlling a switch element of a cell in order to connect the heat control means of the cell to the driver circuit;

determining an actual temperature using the temperature sensor; supplying a data signal from the driver circuit to the heat control means; and

supplying energy corresponding to the data signal.

Each cell is provided with a number of elements. A heating element is provided to perform heating. The heating element may be any suitable heating element, such as a resistive heating element, a peltier element, an infrared heater, and the like. In addition, the cell may comprise a plurality of heating elements. The heating element may further incorporate a cooling element, such as a peltier element. In an embodiment, a cell may comprise a heating element and a cooling element, possibly separately controllable.

A temperature sensor is provided to determine an actual temperature representative of the temperature of the cell at the moment of measurement. The temperature sensor may be any suitable temperature sensor. Further for performing the above method, a switch element is provided in each cell. The function of the switch element is explained in detail below.

The array of temperature controlled cells further comprises at least one driver circuit. The driver circuit is suited to drive at least one temperature controlled cell, i.e. to control the heating element, possibly in response to the actual temperature as determined by the temperature sensor.

In the above method the cell is supplied with a control signal. The control signal controls the switch element of the cell. Due to the control signal, the switch element switches and thereby connects the heat control means of the cell to the driver circuit of the array.

The temperature sensor determines the actual temperature in the temperature-controlled cell. The determination may be performed after the switch element has connected the heat control means to the driver circuit. However, it may as well be a continuous action or may be performed at any other suitable moment during the method. The temperature sensor may output an actual temperature signal. In an embodiment, the actual temperature signal is supplied to the data driver circuit and the data driver circuit determines the data signal corresponding to the actual temperature signal.

The driver circuit is configured to supply a data signal to the heat control means. The data signal represents a setting determined by the driver circuit. The setting may be a heating period indicating a period during which the heating element is to heat the cell; the setting may be a heating power indicating a power with which power the heating element is to heat the cell. Other kind of settings may be suitable as well. In particular, the setting may comprise a set temperature, i.e. a temperature that is to be obtained or maintained in the respective cell. The setting may be stored in a memory element as is described below.

In an embodiment the temperature-controlled cells in an array are arranged in rows and columns. The rows and columns may be substantially perpendicularly arranged, but may as well be arranged otherwise, for example hexagonal or circular arrangements. The cells may be rectangularly shaped corresponding to substantially perpendicularly arranged rows and columns of cells, or the cells may have a different shape such as a hexagonal or circular shape.

In an embodiment, the heat control means comprise a memory element. The memory element may store a setting supplied by the driver circuit. When the setting is stored in the memory element, the control signal may be ended and the memory element may be disconnected from the driver circuit at which point the data signal may be ended. Then, energy may be supplied to the heating element. The amount of energy and/or a period of supply corresponds to the setting stored in the memory element.

Simultaneously, while the heating element is driven corresponding to the setting, the temperature-controlled cells of at least one other row of the array may be supplied with the control signal and with a data signal. Thus, the temperature controlled cells of the array are driven such that they are controlled during short periods of time, while being enabled to supply heat to the cell almost continuously.

The above method enables to use a simple array of temperature controlled cells in which a complex driver circuit is not necessarily comprised in a cell, but may be positioned near a side of the array.

In an embodiment, each cell comprises a first switch element and a second switch element and the method comprises:

supplying a first control signal to the first switch element in order to connect the memory element of the cell to the driver circuit;

supplying a second control signal to the second switch element in order to connect the temperature sensor of the cell to the driver circuit for supplying an actual temperature signal to the driver circuit; and

determining by the driver circuit the data signal to be supplied to the memory element based on the actual temperature signal and a set temperature.

In this embodiment, the temperature sensor is connected to the driver circuit. The driver circuit is thus provided with the actual temperature in the respective temperature controlled cell. The driver circuit is further provided with a set temperature, i.e. a temperature to be obtained or maintained in the respective temperature controlled cell. Based on the actual temperature signal and the set temperature, a setting is determined by the driver circuit and supplied to the cell memory element. Then, the connection may be broken and a connection may be established with another cell.

In an embodiment, the heat control means comprise a control circuit and the method comprises connecting the temperature sensor of the cell to the control circuit for supplying an actual temperature signal to the control circuit. The control circuit may be supplied with the data signal from the data driver circuit and may control the heating element corresponding to the data signal and the actual temperature signal. The data signal may as well be provided to a memory element and the heating element is controlled by the control circuit and by the memory element corresponding to the actual temperature signal and the data signal, respectively.

Further, an array of temperature controlled cells is provided. The cells may be arranged in rows and columns. Each cell has a set of control signal terminals for supplying a control signal to the cell and each cell has a set of data signal terminals for supplying a data signal to the cell. Each cell comprises a heat control means comprising a heating element coupled to an energy source; a switch element coupled to the set of control signal terminals for coupling the heat control means to the data signal terminals in response to the control signal; and a temperature sensor for determining an actual temperature. The array further comprises a data driver circuit connectable to the cell.

In an embodiment the temperature sensor is connectable to the data signal terminals for supplying an actual temperature signal to the driver circuit. Thereto, the cell comprises a first switch for connecting the heat control means to the set of data signal terminals and a second switch for connecting the temperature sensor to the set of data signal terminals.

In an embodiment the heat control means comprise a control circuit, the control circuit being connected to the temperature sensor for supplying a temperature signal to the control circuit and the control circuit being connected to the heating element for controlling the heating element.

The cells of the array may be arranged in rows and columns. The reference to a row or a column is to be regarded as a reference to a line of elements in a first direction or in a second direction, respectively. Reference to a row or column does not explicitly or implicitly refer to any orientation of such a line of elements. A line of elements may be a straight line or may be a differently shaped line. Each cell is a member of one such row and is a member of one such column. Thus, each cell is addressable by addressing a respective row and a respective column.

In a preferred embodiment the array of temperature controlled cells is a biochip. In a more preferred embodiment, this biochip is suitable for performing a chemical reaction.

The array is preferably used for amplification of nucleic acid sequences such as in a PCR reaction.

### BRIEF DESCRIPTION OF THE DRAWINGS

FIG. 1 illustrates a general temperature control scheme using temperature feedback;

FIG. 2A-2B illustrates an active matrix array of temperature controllable cells;

FIG. 3A-3C illustrate embodiments of controllable heating circuits for use in an active matrix array according to FIG. 2A-2B;

FIG. 4A-4B illustrate embodiments of a temperature control circuit for use in an active matrix array according to FIG. 2A-2B;

FIG. 5A-5C illustrate controllable heating circuits with in-cell temperature feedback;

FIG. 6A-6B illustrate embodiments of a controllable heating circuit according to FIG. 5A-5C for use in an active matrix array according to FIG. 2A-2B;

### DETAILED DESCRIPTION OF EXAMPLES

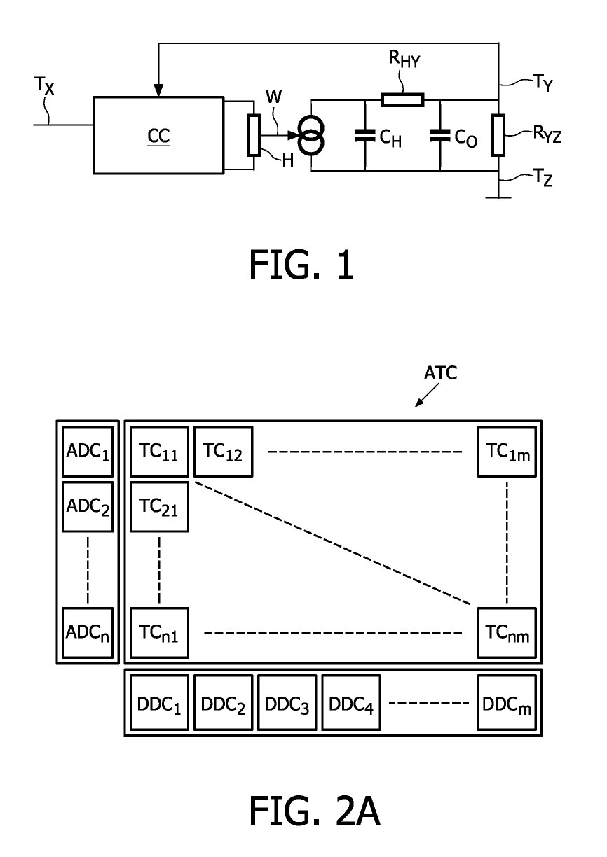

FIG. 1 shows an embodiment of a general control model for thermal control of a cell of an array. A control circuit CC drives a heating element H. In the heating element H, electrical power is converted to heat. In the thermal control model the generated heat is indicated as a heat current W. A heat capacity of an object may be represented by an analogue of a capacitor and the inverse of a thermal conductivity i.e. a heat resistance. Hence, the control model comprises a heat capacity C_{H} and a thermal resistance R_{HY} representing the heater H. The electrical heater H is thermally connected to a sample having a heat capacity C_{O}. The sample loses heat to the environment, having a temperature T_{Z}, through the thermal resistance R_{YZ} of its insulation.

In operation, a sample temperature T_{Y} is fed back to the control circuit CC. In response to the actual sample temperature T_{Y} and a set temperature T_{X} the temperature control circuit CC adjusts the power dissipated in the heating element H, and thus the heat current W such that a difference between the actual temperature T_{Y} and the set temperature T_{X} is as small as possible.

The stability and accuracy of the control method depends on the type of control. A number of feedback control types may be employed:

1) On-Off control—Heating is turned off when the sample temperature T_{Y} is above the set temperature T_{X} and heating is turned on when the sample temperature T_{Y} is below the set temperature T_{X}. This method is slow and has low accuracy and can have significant overshoot and undershoot.
2) Proportional control—Heating current W is applied proportional to the difference between the actual sample temperature T_{Y} and the set temperature T_{X}. This removes the temperature cycling of “On-Off control” and gives moderate temperature control with a smoother action. Thus:

W=P(T_{X}−T_{Y})

where P is the proportional gain. If the sample temperature T_{Y} is higher than the set temperature T_{X}, the heating is turned off.
3) Proportional Differential control—Differential control adds a damping factor which can be made critical to achieve the set temperature T_{X} without overshoot and ringing, or undershoot and slow response. This method gives improved accuracy but is less noise tolerant and gives a steady state offset:

$$W = P ( ( {T}_{X} - {T}_{Y} ) + D \frac{d}{d t} ( {T}_{X} - {T}_{Y} ) )$$

where D is a damping factor.

4) Proportional Integral Differential control—Compared to Proportional Differential Control, a further integral control is added to correct for a steady state offset. Heating is changed until a time averaged response goes to zero:

$$W = P ( ( {T}_{X} - {T}_{Y} ) + D \frac{d}{d t} ( {T}_{X} - {T}_{Y} ) + I \int ( {T}_{X} - {T}_{Y} ) d t )$$

where I is an integral gain.
5) Phase control—Each of the above types of temperature control may be applied in a multiple phase approach of temperature control. In a multiple phase approach, the temperature control is divided in a number of phases. Various types of temperature control may be applied, and per phase the parameters {P,I,D} may be varied. For example, a 3-phase fast temperature control with high accuracy and negligible overshoot may consist of an ‘approach’ phase, a ‘hand-over’ phase and a ‘control’ phase. During the ‘approach’ phase the temperature T_{Y} is moved fastly (e.g. maximum ramp) towards the set temperature T_{X}. To prohibit overshoot, the ‘hand-over’ phase is enabled as soon as the actual temperature T_{Y} is a predetermined temperature difference away from the set temperature T_{X}. During the ‘hand-over’ phase the temperature is brought to the set temperature T_{X}. Thereafter, the ‘control’ phase is enabled to stabilize the actual temperature T_{Y} at the set temperature T_{X}.

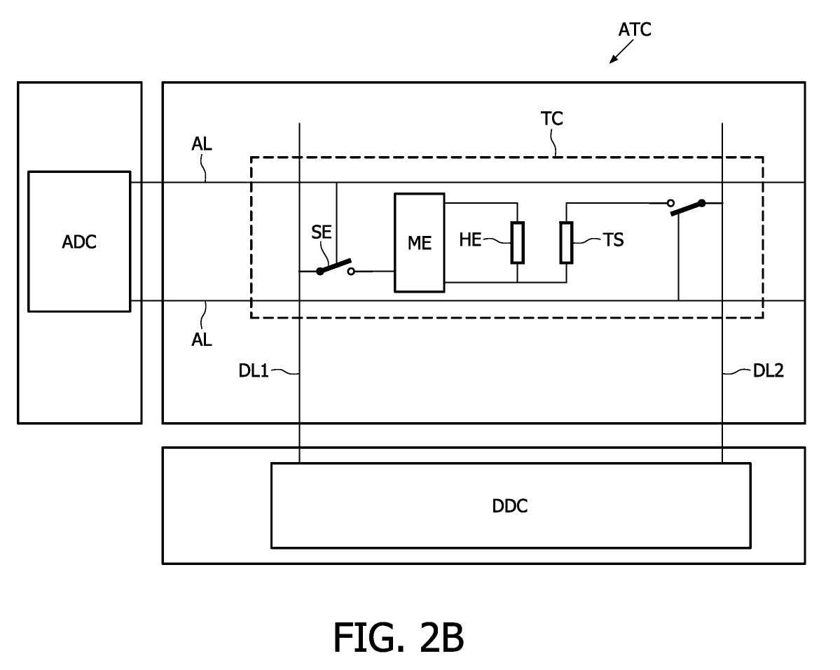

The above-described temperature control methods may be employed to control the temperature of a cell of an array of cells. Such an array of temperature controlled cells are known in the art. Each cell may function as a chemical reaction chamber, for example for use in a so-called bio-chip. FIGS. 2A and 2B illustrate an array of temperature controlled cells according to an embodiment of the present invention. In FIG. 2A an array ATC of n×m temperature controlled cells TC is shown. The cells TC are arranged in n rows and m columns. The rows and columns are arranged perpendicularly, but may as well be arranged differently as described above. Each row of cells TC is connectable to an address driver circuit ADC and each column of cells TC is connectable to a data driver circuit DDC. The function of the address driver circuit and the data driver circuit is elucidated hereinafter. Thus, a first row of cells TC₁₁-TC₁ₘ is connected to a first address driver circuit ADC₁; a first column of cells TC₁₁-TCₙ₁ is connected to a first data driver circuit DDC₁. Likewise, a temperature controlled cell TCₙₘ is connectable to a n^{th} address driver circuit ADCₙ and is connectable to a m^{th} data driver circuit DDCₘ.

FIG. 2B illustrates a temperature controlled cell TC in more detail. The temperature controlled cell TC comprises a heating element HE, an optional memory element ME, a switch element SE and a temperature sensor TS. The memory element ME is connectable to the data driver circuit DDC via the switch element SE that is connected to and controlled by the address driver circuit ADC. The temperature sensor TS is connectable to the data driver circuit DDC as well, simultaneously with the memory element ME, controlled by the address driver circuit ADC. Thus, at least one address line AL is provided between the cell TC and the address driver circuit, and at least two column driver circuit data lines DL1 and DL2 are provided between the data driver circuit DDC and the cell TC. One data line DL1 may be employed for supplying data from the data driver circuit DDC to the memory element ME of the cell TC, and the other data line DL2 may be employed to supply temperature feedback data from the cell TC to the data driver circuit DDC.

The heating element HE may be any suitable heating element, such as a resistor, a peltier element, an infrared heater, and the like. The temperature sensor may be any suitable temperature sensor such as a reversed bias PN junction diode or transistor providing a leakage current or a band-gap temperature sensor as known in the art.

In operation, referring to FIGS. 2A and 2B, one row of temperature controlled cells TCN₁-TCNₘ is addressed by the respective address driver circuit ADC_{N}, thereby each cell TC of the N^{th} row is connected to the respective data driver circuits DDC₁-DDCₘ. The cells TC of the other rows are not addressed and are therefore not connected to the data drivers DDC. Thus, when addressed by the respective address driver circuit ADC, the memory element ME of each cell TC of the corresponding row is connected to the corresponding data line DL1 and the temperature sensor TS is connected to the corresponding data line DL2. In the absence of a memory element, the heating element of each cell TC of the corresponding row is connected to the corresponding data line DL1.

When addressed the temperature controlled cell TC is connected to the data driver circuit DDC. The data driver circuit DDC comprises the circuitry for temperature control. The actual cell temperature (FIG. 1: T_{Y}) is supplied to the data driver circuit DDC and the data driver circuit DDC is configured to determine a temperature difference between the actual cell temperature and a set temperature. Then, corresponding to the determined temperature difference the data driver circuit DDC supplies a heating setting to the memory element ME of the addressed cell TC indicating an amount of heating power to be delivered to the temperature controlled cell TC. After the heating setting is stored in the memory element ME, the address driver circuit ADC may disconnect the cell TC from the data driver circuit DDC and then another address driver circuit ADC may address another row of temperature controlled cells, for example a next row of cells TC_{(N+1)1}-TC_{(N+1)m}. In the period that the temperature controlled cell TC is not connected to the data driver circuit DDC, the cell TC may be heated by the heating element HE based on the setting stored in the memory element ME.

These method steps may be repeated for each row of cells TC and thus each cell TC of the array ATC may be temperature controlled using a control circuit (data driver circuit DDC) without requiring that each cell TC is provided with such a complex control circuit. The period required to control all rows is referred to hereinafter as a field period. Thus, in one field period, all rows are addressed and controlled once per field period. It is noted that the connection of the data driver circuit DDC and the address driver circuit ADC with respect to the columns and rows may be exchanged.

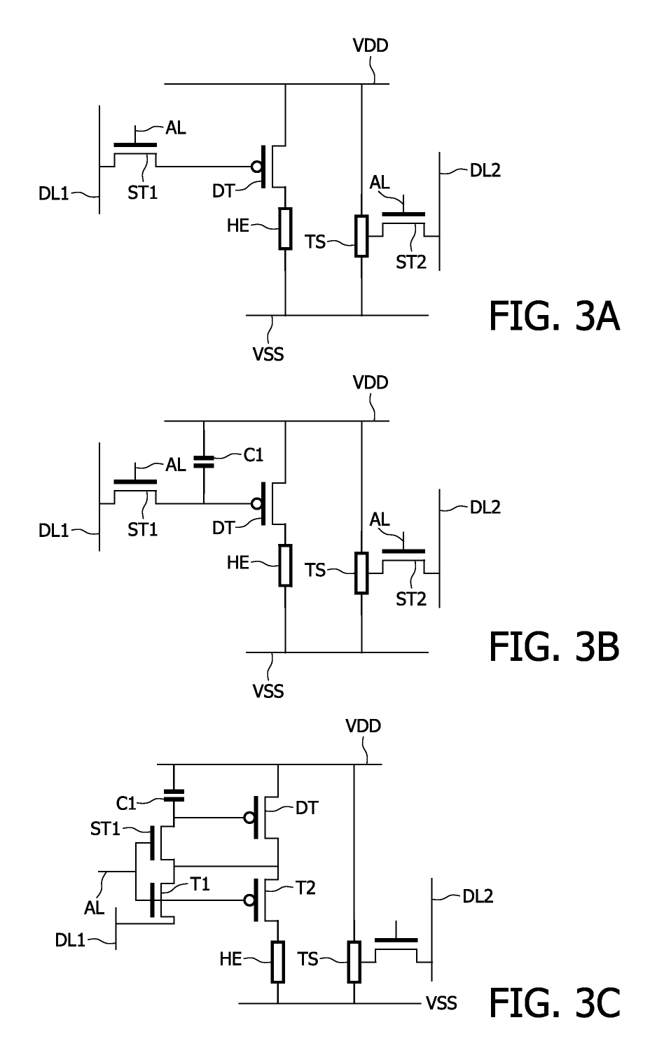

FIG. 3A shows an embodiment of a temperature control circuit without a memory element for use in a cell of an array according to the present invention. The temperature control circuit is coupled to an energy source via a first and a second energy source terminal VDD, VSS, respectively. The gate of a drive transistor DT is connected to a drain of a first switch transistor ST1, of which a source is connected to a first data line DL1 and a gate is connected to the address line AL. The source of the drive transistor DT is connected to the first energy source terminal VDD and the drain is connected to the heating element HE. The heating element HE is further connected to the second energy source terminal VSS.

FIG. 3B shows an embodiment of a temperature control circuit comprising a memory element for use in a cell of an array according to the present invention. The temperature control circuit is coupled to an energy source via a first and a second energy source terminal VDD, VSS, respectively. A capacitor C1 is coupled between the first energy source terminal VDD and a gate of a drive transistor DT. The gate of the drive transistor DT is further connected to a drain of a first switch transistor ST1, of which a source is connected to a first data line DL1 and a gate is connected to the address line AL. The source of the drive transistor DT is connected to the first energy source terminal VDD and the drain is connected to the heating element HE. The heating element HE is further connected to the second energy source terminal VSS.

In both FIG. 3A and FIG. 3B, a second switch transistor ST2 is connected to a second data line DL2 and the temperature sensor TS. The gate of the second switch transistor ST2 is connected to an address line AL2. This may be the same address line AL as to which the first switch transistor ST1 is connected or may a separate address line.

When the corresponding address driver circuit ADC addresses the cell, the switch transistors ST1 and ST2 are switched conductive, connecting the temperature sensor TS and the memory element, i.e. capacitor C1 (FIG. 3B), or the gate of the driving transistor of the heating element DT (FIG. 3A) to the data driver circuit DDC. In response to the actual cell temperature as determined by the temperature sensor TS, the data driver circuit DDC supplies a heating setting to the capacitor C1 (FIG. 3B), or the gate of the driving transistor of the heating element DT (FIG. 3A). Thereby, the capacitor C1 (FIG. 3B) is charged to a predetermined level. In the embodiment of FIG. 3A, the gate of the driving transistor DT of the heating element HE is switched conductive, or not, in order to supply energy to the heating element HE for heating the temperature-controlled cell, or not.

If the temperature control circuit as shown in FIG. 3A-3B is required to deliver as much power as possible to the heating element HE, minimal power should be lost in the drive transistor DT. This requires the drive transistor DT to be driven in a switching mode so that its drain-source voltage is minimal. Therefore, in such an embodiment, the heating element HE can only be either on or off. The temperature may then be controlled by a length of time during which current is driven to the heating element HE. The data driver circuit DDC may control this once per field period. As a result the shortest period that the heating element HE can be on is one field period.

Alternatively, if power loss across the drive transistor DT is acceptable, the drive transistor DT may be driven as a current source and analogue data voltages controlling the current may be stored on the capacitor C1 over a field period. However, the voltage at the drain node of the drive transistor DT may not be well defined so the power, and hence heat produced and delivered, may not be well controlled. Methods such as current programming and threshold voltage measurement may be useful for producing more accurate power delivery enabling uniform power delivery across an array of cells.

FIG. 3C shows a current mirror circuit that may be used to compensate for threshold and mobility variations of the drive transistor DT and hence enable more uniform power delivery. The circuit shown in FIG. 3C comprises two additional transistors T1 and T2 (compared to the circuit shown in FIG. 3B). The current mirror circuit shown in FIG. 3C is known in the art and detailed explanation of its operation is therefore omitted here. In general, the control circuit of FIG. 3C stores a setting in the form of currents by charging the capacitor C1. At the address period the gate of the switch transistor ST1 is high, so current is drawn through the drive transistor DT, thereby charging the capacitor C1 to a voltage necessary to deliver a corresponding predetermined current. After the address period, the predetermined current is delivered to the heating element HE. Since the delivered power (P) is a function of the current (I) and the heating element resistance (R) (i.e. P=I² R) and assuming that the heating element resistance (R) is a constant, uniform heating power may be delivered to the cell.

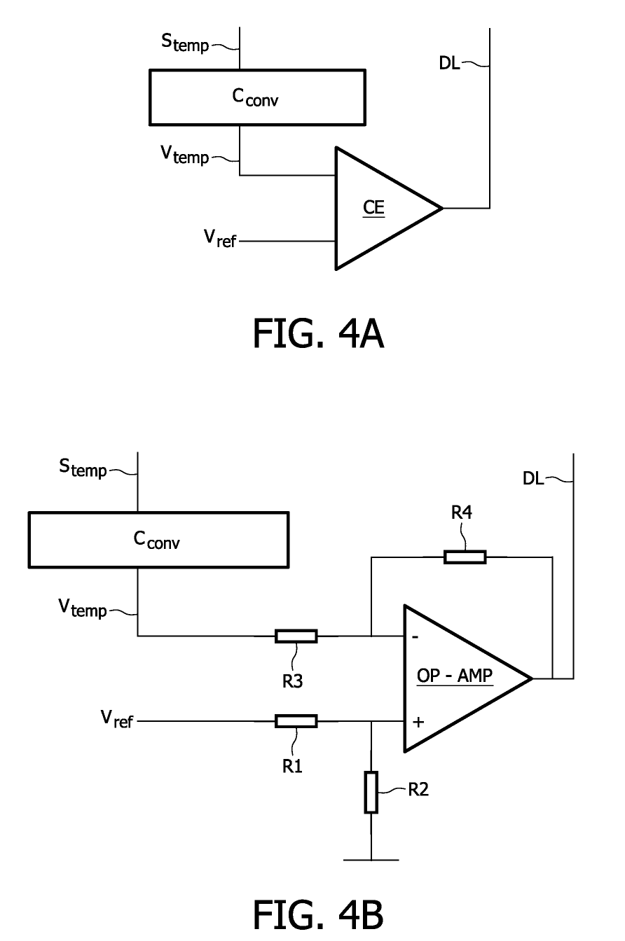

FIGS. 4A and 4B illustrate embodiments of data driver circuits DDC for use in the array as shown in FIGS. 2A and 2B. The data driver circuit DDC, i.e a temperature control circuit, may be connected as a row driver circuit or as a column driver circuit. In the illustrated embodiments, it has been assumed that the temperature control driver circuit is the column driver circuit. However, it may as well be the row driver circuit.

FIG. 4A illustrates an embodiment for on/off control as described in relation to FIG. 1. A comparator element CE has a reference voltage V_{ref} as an input representing a set temperature. The comparator element CE has as a further input a signal supplied by the temperature sensor of the temperature controlled cell. The signal may be amplified and/or converted by a suitable circuit C_{conv} to convert the temperature signal Sₜₑₘₚ to a suitable temperature voltage Vₜₑₘₚ. An output of the comparator element CE is supplied to the memory element of the cell. In this embodiment, the voltage/current from the temperature sensor is thus compared with a reference voltage V_{ref}, i.e. the set temperature. If the voltage/current is less than the reference voltage V_{ref}, heating is started or continued; if the voltage/current is higher than the reference voltage V_{ref}, heating is stopped. This embodiment of a data driver circuit is particularly suited to drive the circuit shown in FIG. 3B in a digital mode.

FIG. 4B illustrates an embodiment of a data driver circuit DDC suitable for proportional control. The temperature signal Sₜₑₘₚ supplied by the temperature sensor of the cell may be amplified and converted to a suitable voltage Vₜₑₘₚ by a suitable converter circuit C_{conv}. A reference voltage V_{ref} is provided. The suitable temperature voltage Vₜₑₘₚ and the reference voltage V_{ref} are supplied to an operational amplifier OP-AMP through suitably selected resistors R1-R4, providing a predetermined gain. Selection of the resistances of the resistors R1-R4 in order to obtain a desired gain is known in the art and is not further explained here. An output of the operational amplifier circuit is supplied to the memory element of the temperature controlled cell.

The above-described embodiments operate in an intermittent manner, hence the temperature data and the memory data are updated once per field period. In another embodiment of the present invention, the temperature feedback may be performed within the temperature controlled cell. This may provide more accurate temperature control, since the temperature feedback is virtually continuous, while in the above-described embodiments, the feedback is only once per field period. The memory element may then store a set temperature that may be updated once per field period. The actual temperature control in response to the set temperature is performed within the cell.

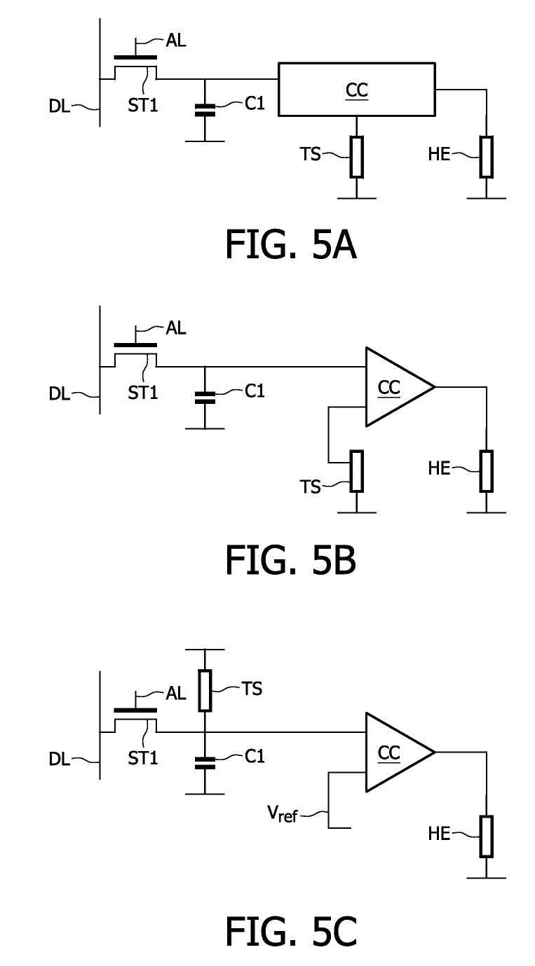

FIG. 5A illustrates a general in-cell control scheme. An input of an in-cell control circuit CC is connected to a capacitor C1, functioning as a memory element. The capacitor C1 is further connected to a switching element, i.e. transistor ST1. The transistor ST1 is further connected to a data line DL e.g. connected to a data driver circuit DDC and the gate of the transistor ST1 is connected to an address line AL e.g. connected to an address driver circuit ADC. Another input of the control circuit CC is connected to a temperature sensor TS. An output of the control circuit CC is connected to a heating element HE. In response to the input of the reference voltage supplied by the capacitor C1 and the voltage supplied by the temperature sensor TS, the control circuit CC controls the heating element HE.

FIG. 5B illustrates an embodiment for on/off control of the heating element HE. The control circuit comprises a comparator element CE. During an address period, in which the data line DL is connected to the capacitor C1 through the switch transistor ST1 as controlled through the address line AL, the capacitor C1 may be charged to a voltage level corresponding to a set temperature. After the address period, the comparator element CE compares the reference voltage of the capacitor C1 and the output voltage of the temperature sensor TS. If the voltage of the temperature sensor is lower than the reference voltage, the heating element HE is switched on, otherwise the heating element HE is switched off.

In another embodiment as shown in FIG. 5C, the temperature sensor TS is connected to the capacitor C1 and the comparator element CE has a reference voltage V_{ref} as an input instead of the temperature sensor voltage (compared to the circuit as illustrated in FIG. 5B). The temperature sensor TS generates a current that is supplied to the capacitor C1, thereby the voltage of the capacitor C1 increases. Eventually the voltage of the capacitor C1 reaches the reference voltage V_{ref} resulting in switching the heating element HE off.

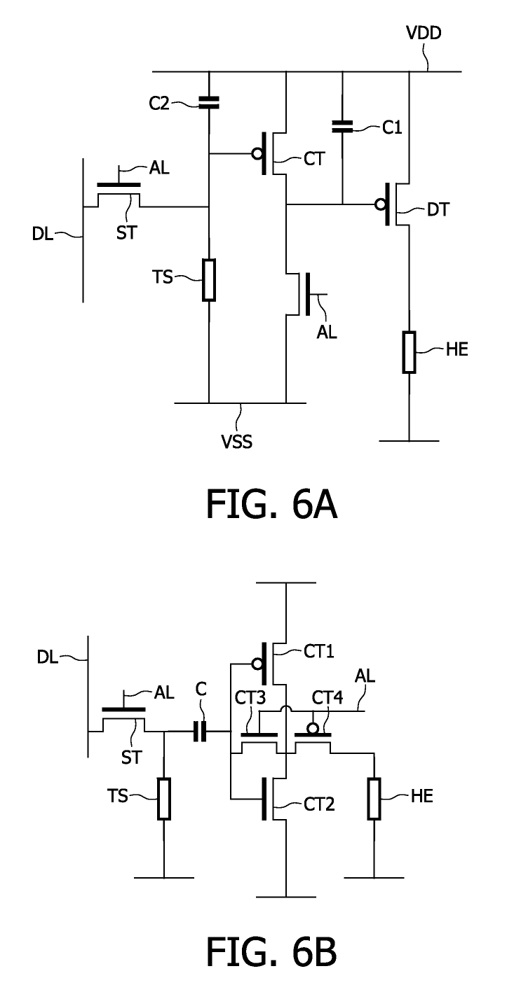

FIG. 6A shows an embodiment of the above-described circuit. The heating element HE is connected between a first energy source terminal VSS and a drive transistor DT. The drive transistor DT is further connected to a second energy source terminal VDD and its gate is connected to a first capacitor C1 and a drain of a control transistor CT. The first capacitor C1 is further connected to the first energy source terminal VDD. The source of the control transistor CT is as well connected to the first energy source terminal VDD. A second capacitor C2 is connected between the gate of the control transistor CT and the first energy source terminal VDD. The gate of the control transistor CT is further connected to a switch transistor ST, which may connect the gate of the control transistor to a data line DL depending on a signal on an address line AL which is connected to the gate of the switch transistor ST. The temperature sensor TS is connected between the second energy source terminal VSS and the second capacitor C2.

In the embodiment shown if FIG. 6A, the threshold voltage (Vt) of the drive transistor acts as the reference voltage. Below the threshold voltage (Vt), the transistor acts as an insulator, above the threshold voltage, the transistor acts as a conductor. The circuit delivers power to the heater via the drive transistor DT that is held in its on state by the voltage stored on the first capacitor C1. The first capacitor C1 is charged at the address period from VSS. Also at the address period, the second capacitor C2 is charged with a data voltage that represents the set temperature. The data voltage on the second capacitor C2 is initially sufficiently low to ensure that the control transistor CT is initially in its off state. ‘Initially’ refers to the moment directly after completing the address period. After the address period, power is delivered to the heating element HE which as a result starts to heat up. The temperature sensor TS generates a current proportional to the temperature and charges C2 so that eventually the control transistor CT turns on to discharge the first capacitor C1. As a result, the heating element HE is turned off. Thus, power is delivered to the heating element in a pulse width modulation fashion. During the heating period, i.e. the period not being the address period, the power may be applied for a fraction of the heating period i.e. a duty period.

To increase the temperature, the data voltage representing the set temperature, i.e. the voltage on the second capacitor C2, is increased causing the duty period to initially increase thereby delivering more power to the heating element. As the temperature increases the temperature sensor TS starts to produce more current charging the second capacitor C2 to the threshold voltage of the control transistor CT at a higher rate. Thus, the duty period is shortened. Eventually, a stable steady-state at the desired set temperature is achieved. To reduce the temperature, the data voltage on the second capacitor C2 is reduced causing the duty period to shorten or become zero. As the temperature drops, the current in the temperature sensor TS reduces and the duty period starts to increase. A stable steady-state will eventually be reached at the desired lower temperature.

As described above, the heating element HE may be switched on during any period of time (duty period) required for heating the cell to the desired set temperature. Thus, a better accuracy may be achieved compared to the control method as described in relation to FIG. 2A-4B.

FIG. 6B illustrates an embodiment wherein the heating element HE is controlled using proportional control. An inverter circuit is used to provide a desired gain. The control circuit comprises a switch transistor ST connected to a data line DL (source) and an address line AL (gate). The drain of the switch transistor ST is connected to the temperature sensor TS and a capacitor C. The capacitor C is further connected to the gate of a first and a second control transistor CT1-CT2 comprised in the inverter circuit. A third and a fourth control transistor CT3-CT4 are provided between the inverter circuit and the heating element HE. The gates of the third and the fourth control transistors CT3-CT4 are connected to the address line AL and these control transistors CT3-CT4 operate as switches. The operation of the inverter circuit is known in the art and is therefore not discussed in detail here. In general, if the gate voltage of the control transistor CT2 is high, the voltage over the heating element HE is low, and if the gate voltage of the control transistor CT2 is low, the voltage over the heating element HE is high.

At the address period, data, i.e. a voltage, is stored on capacitor C and the inverter is held at a mid-point in this period, so the capacitor C can be charged and any inverter offsets cancelled. The current to the heating element HE is also switched off in this period. After the address period (and assuming the set temperature is higher than the actual temperature) the capacitor C is switched to the temperature sensor voltage, which is low at that moment, and current then flows to the heating element HE so the temperature sensor voltage rises, thereby reducing the current to the heating element HE. Then, with an appropriately designed system, a stable temperature may be achieved.

The gain in the circuit of FIG. 6B may not be accurately controlled. To provide a more accurately controlled gain, a full operational amplifier may need to be implemented and resistors may then be used to control the differential gain. Such a circuit would be similar to the circuit illustrated in FIG. 4B.

More sophisticated systems such as systems with integration and differentiation as described above in relation to FIG. 1, may need further operational amplifiers to be implemented. However, such circuits may be designed by a person skilled in the art and is therefore not described and shown.

In the above description of the drawings, reference is made to transistors in general. In practice, the temperature controlled cell-array is suited to be manufactured using Low Temperature Poly-Silicon (LTPS) Thin Film Transistors (TFT). Therefore, in an embodiment, the transistors referred to above may be TFT's. In particular, the array may be manufactured on a large area glass substrate using LTPS technology, since LTPS is particularly cost effective when used for large areas.

Further, although the present invention has been described with regard to low temperature poly-Si (LTPS) based active matrix device, amorphous-Si thin film transistor (TFT), microcrystalline or nano-crystalline Si, high temperature poly SiTFT, other anorganic TFTs based upon e.g. CdSe, SnO or organic TFTs may be used as well. Similarly, MIM, i.e. metal-insulator-metal devices or diode devices, for example using the double diode with reset (D2R) active matrix addressing methods, as known in the art, may be used to develop the invention disclosed herein as well.

1. Method of driving an array of temperature controlled cells, each cell comprising a heat control means comprising a heating element, a switch element, and a temperature sensor, and the array further comprising a driver circuit, the method comprising:
supplying an address signal for controlling a switch element of a cell in order to connect the heat control means of the cell to the driver circuit;
determining an actual temperature using the temperature sensor;
supplying a data signal from the driver circuit to the heat control means; and
supplying energy corresponding to the data signal.

2. Method according to claim 1, wherein the data signal comprises a set temperature.

3. Method according to claim 1, wherein the method further comprises:
supplying an output of the temperature sensor to the driver circuit; and
determining within the driver circuit the data signal dependent upon the output of the temperature sensor.

4. Method according to claim 3, wherein the cell comprises a first switch element and a second switch element, the method comprising:
supplying a first address signal to the first switch element in order to connect the heat control means to the driver circuit for supplying the data signal to the heat control means; and
supplying a second address signal to the second switch element in order to connect the temperature sensor of the heat control means to the driver circuit for supplying an actual temperature signal to the driver circuit.

5. Method according to claim 1, wherein the heat control means further comprises a memory element and the data signal comprising a setting, the method further comprising:
connecting the memory element of the heat control means to the driver circuit in response to the control signal;
storing the setting comprised in the data signal in the memory element; and supplying energy corresponding to the setting stored in the memory element.

6. Method according to claim 1, wherein the array of temperature controlled cells are arranged in rows and columns, the method further comprising:
supplying the control signal to each cell of a row of cells for controlling the switch element of the cell in order to connect the heat control means of the cell to the driver circuit.

7. Method according to claim 1, wherein the heat control means comprise a control circuit and the method comprises:
connecting the temperature sensor of the cell to the control circuit for supplying an actual temperature signal to the control circuit.

8. Method according to claim 7, wherein the temperature signal modifies the setting as stored on the memory element.

9. Array of temperature controlled cells, the array comprising a data driver circuit and each cell comprising heat control means, the heat control means comprising:
a heating element coupled to an energy source;
a switch element coupled to an address signal terminal for coupling the heat control means to a data signal terminal in response to the address signal, the data signal terminal being connected to the data driver circuit; and
a temperature sensor for determining an actual temperature.

10. Array according to claim 9, wherein the heat control means further comprises: a memory element connectable to the data signal terminals for receiving a setting to be stored, the setting being comprised in a data signal.

11. Array according to claim 10, wherein the memory element is a capacitor coupled to a gate of a drive transistor, the drive transistor being configured to control a current to be supplied to the heating element.

12. Array according to claim 9, wherein the temperature sensor is connectable to the driver circuit for supplying an output of the temperature sensor to the driver circuit.

13. Array according to claim 12 wherein the cell is provided with a first switch element for connecting the heat control means to the data signal terminals in response to a first address signal and a second switch element for connecting the temperature sensor to the data signal terminals in response to a second address signal.

14. Array according to claim 12, wherein the data driver circuit is configured to determine a heating setting in response to an actual temperature supplied by the temperature sensor and a set temperature and to supply the heating setting to the heat control means through the data signal.

15. Array according to claim 14, wherein the data driver circuit comprises a comparator element for comparing the actual temperature and the set temperature to determine whether the actual temperature is higher or lower than the set temperature and being configured to control the heat control means accordingly.

16. Array according to claim 9, wherein the data driver circuit comprises an operational amplifier, a first resistor connected between a set temperature terminal and a first input of the operational amplifier, a second resistor connected between ground and the first input terminal, a third resistor connected between an actual temperature terminal and a second input terminal of the operational amplifier and a fourth resistor connected between the second input terminal and an output terminal of the operational amplifier, such that an output signal generated on the output terminal is proportional to a difference between the actual temperature and the set temperature.

17. Array according to claim 9, wherein the cells are arranged in rows and columns,
each row of cells having a set of address signal terminals for supplying an address signal to each cell of the row of cells; and
each column of cells having a set of data signal terminals for supplying a data signal to a cell in the column of cells.

18. Array according to claim 9, wherein the heat control means comprise a control circuit, the control circuit being connected to the temperature sensor for supplying a temperature signal to the control circuit and the control circuit being connected to the heating element for controlling the heating element.

19. Array according to claim 18, wherein the temperature sensor is coupled to the memory element in order to modify the setting as stored on the memory element in response to the actual temperature.

20. Array according to claim 9 wherein the array is a biochip.

21. Use of an array according to claim 9 for the amplification of nucleic acids.
